(19) United States
(12) Patent Application Publication
(10) Pub. No.: US 2002/0068986 A1
(43) Pub. Date: Jun. 6, 2002

(54) Adaptation of audio data files based on personal hearing profiles

(76) Inventor: Ali Mouline, Mountain View, CA (US)

Correspondence Address: HAYNES BEFFEL & WOLFELD LLP, P O BOX 366, HALF MOON BAY, CA, 94019, US

(21) Appl. No.: 09/728,623

(22) Filed: Dec. 1, 2000

Related U.S. Application Data
(60) Provisional application No. 60/168,290, filed on Dec. 1, 1999.

(51) Int. Cl. G06F 17/00; H04R 29/00; A61B 5/00

(52) U.S. Cl. 700/94; 381/60; 600/559

(57) ABSTRACT

Methods and systems for high quality computer based adaptation of audio data are shown. Adaptation, delivery of audio data, and testing of user's hearing abilities can occur on computers or computer networks such as the Internet Adaptation can compensate for frequency dependent and audio masking impairments. The audio to be adapted can include real-time streaming digital data or static data files. Standard digital audio formats are supported.

### CROSS REFERENCE TO RELATED APPLICATION

The present application claims the benefit of priority from U.S. Provisional Patent Application No. 60/168,290, entitled “System for Providing Uniquely Adapted Internet Audio” filed on Dec. 1, 1999, which is incorporated by reference herein.

### BACKGROUND OF THE INVENTION

1. Field of the Invention

The present invention relates generally to the modification of audio signals on computing systems and more specifically to the modification of audio signals for the purpose of compensating for hearing impairments.

2. Background

Hearing impairments may result in a variety of clinical manifestations. For example, a person may have adequate hearing in the 20 to 2000 Hz range and rapidly diminishing sensitivity from 2000 to 20,000 Hz. In some cases, people can be overly sensitive to a narrow set of frequencies; for example, the pain threshold may be reduced from a typical 120 dB to much lower levels. Some people also experience a shift in perceived frequencies. Low frequency sounds can be heard as high frequency sounds or visa versa. Finally, people can have abnormal audio masking profiles. Audio masking is a normal process in which strong sounds reduce sensitivity to closely related frequencies or sounds that occur within a short temporal period. In abnormal conditions, the width or height of the masking thresholds may be unusually large.

Each of these conditions represents hearing impairments that cannot be compensated for by simply increasing the overall volume of the sound. Compensation must therefore be made as a function of signal frequency or temporal relationships.

3. Description of the Prior Art

Prior art is found in four fields: hearing aids, telecommunications, hearing testing, and audio signal processing. Many prior art references encompass two or more of these fields.

Gharib et al. (U.S. Pat. No. 3,571,529), Bottcher et al. (U.S. Pat. No. 3,764,745), Kryter (U.S. Pat No. 3,894,195), Rohrer et al. (U.S. Pat. No. 3,989,904), Strong et al. (U.S. Pat. No. 4,051,331), Mansgold et al. (U.S. Pat. No. 4,425,481), Zollner et al. (U.S. Pat. No. 4,289,935), Engebretson et al. (U.S. Pat. No. 4,548,082), Slavin (U.S. Pat. No. 4,622,440), Levitt et al. (U.S. Pat. No. 4,731,850), Nunley et al. (U.S. Pat. No. 4,791,672), Bennett (U.S. Pat. No. 4,868,880), Cummins et al. (U.S. Pat. No. 4,887,299), Anderson et al. (U.S. Pat. No. 4,926,139), Williamson et al. (U.S. Pat. No. 5,027,410), Zwicker et al. (U.S. Pat. No. 5,046,102), Kelsey et al. (U.S. Pat. No. 5,355,418), Miller et al. (U.S. Pat. No. 5,406,633), Stockham et al. (U.S. Pat. No. 5,500,902), Magotra et al. (U.S. Pat. No. 5,608,803), Vokac (U.S. Pat. No. 5,663,727), Engebretson et al. (U.S. Pat. No. 5,706,352), Anderson (U.S. Pat. No. 5,721,783), Ishige et al. (U.S. Pat. No. 5,892,836), Salmi et al. (U.S. Pat. No. 5,903,655), Stockham et al. (U.S. Pat. No. 6,072,885), Melanson et al. (U.S. Pat. No. 6,104,822), Schneider (WO9847314A2), Hurtig et al. (WO9914986A1), and Leibman (EP329383A3) disclose hearing aid devices that perform in a frequency dependent manner. Several of these focus on the relative enhancement of frequencies associated with speech. Enhancement may be accomplished through a variety of programmable amplifiers or filters or operations in the frequency domain.

Hearing aids are limited in their processing power, programmability, and convenience. Lack of processing power results in adaptation over a reduced frequency range and limits the quality of the audio output. Programmability is desirable when a user's hearing impairments change over time. While simple adjustments, such as optimization for voice or music, can be made by a user, there is no system in the prior art for users to simply adjust for frequency dependent impairments. Finally, hearing aids can only apply adaptation to an audio signal after it has reached the user as sound waves. Background noises are, therefore, also affected and possibly enhanced by the adaptation process. It would be advantageous to apply adaptation prior to arrival of sound at the user.

Terry et al. (U.S. Pat. No. 5,388,185), Dejaco (WO9805150A1), Nejime (U.S. Pat. No. 5,794,201), and Deville et al. (U.S. Pat. No. 6,094,481) disclose methods for adjusting the intensity of sound delivered over a telephone network as a function of frequency and a consumer's hearing characteristics. These systems are limited by differences between audio testing systems and typically inferior telephone speakers. They also lack convenient means for relaying a user's particular hearing prescription to telephone network databases or later editing that data and the prescription changes.

Cannon et al. (U.S. Pat. No. 3,718,763), Hull (U.S. Pat. No. 4,039,750), Bethea et al. (U.S. Pat. No. 4,201,225), Killion (U.S. Pat. No. 4,677,679), Shennib (U.S. Pat. No. 5,197,332), Clark et al. (U.S. Pat. No. 5,928,160), and Garrett (WO9931937A1) disclose systems for testing hearing. These systems all require special equipment with limited availability.

Hoarty (U.S. Pat. No. 5,594,507), Galbi (U.S. Pat. No. 5,890,124), Smyth et al. (U.S. Pat. No. 5,956,674), Smyth et al. (U.S. Pat. No. 5,974,380), Smyth et al. (U.S. Pat. No. 5,978,762), Gentit (U.S. Pat. No. 5,987,418), Malvar (U.S. Pat. No. 6,029,126), Nishida (U.S. Pat. No. 6,098,039), and The Digital Signal Processing Handbook (Vijay K. Madisetti and Douglas B. Williams, IEEE, CRC Press 1997) disclose audio encoding or decoding systems that take advantage of audio masking effects. These references demonstrate the depth to which audio masking is understood.

Alverez-Tinoco, (WO9851126A1), and Unser et al. (“B-spine signal processing:Part II—efficient design and applications”, IEEE Trans. Signal Processing, vol 41, no2, pp. 834-848.) disclose general methods for signal processing.

### SUMMARY

Systems and methods are described for assisting a hearing deficient listener by adapting audio according to the listener's personal auditory capability. The system includes a database for storage of listener audio profiles, which are typically described in terms of threshold and limit parameters for a plurality of audible frequencies. Upon utilization of the system by a listener, an adaptation engine operates by accessing the audio profile and retrieving an audio file selected by the listener. The adaptation engine modifies the audio file based on the listener's audio profile, thus assisting the listener in perceiving the audio. The modification is performed generally through a process involving audio data conversion, transformation, and scaling to the listener's needs. The scaling may include frequency shifting, frequency filtering, frequency masking compensation, and adaptive signal processing. The adapted audio can subsequently be stored and transmitted to the listener for presentation.

A preferred operating environment includes a client computer and server computer communicating through a network such as the Internet, wherein the listener utilizes the client computer to access the service provided by the server computer. Alternative embodiments contemplate that the adaptation process may occur at either the client or server computer.

### BRIEF DESCRIPTION OF THE DRAWINGS

FIG. 1 depicts an exemplary operating environment of an embodiment of the invention.

FIG. 2 shows a flow diagram of the execution of an embodiment of the invention.

FIG. 3 depicts the components of an adaptation system, according to an embodiment of the invention.

FIG. 4 illustrates principal steps of an embodiment of the invention.

FIG. 5 depicts alternative methods of collecting or accessing personal hearing data in accordance with embodiments of the invention.

FIG. 6 depicts details of systems that can be used to generate hearing data according to alternative methods of FIG. 5.

### DETAILED DESCRIPTION OF THE EMBODIMENTS

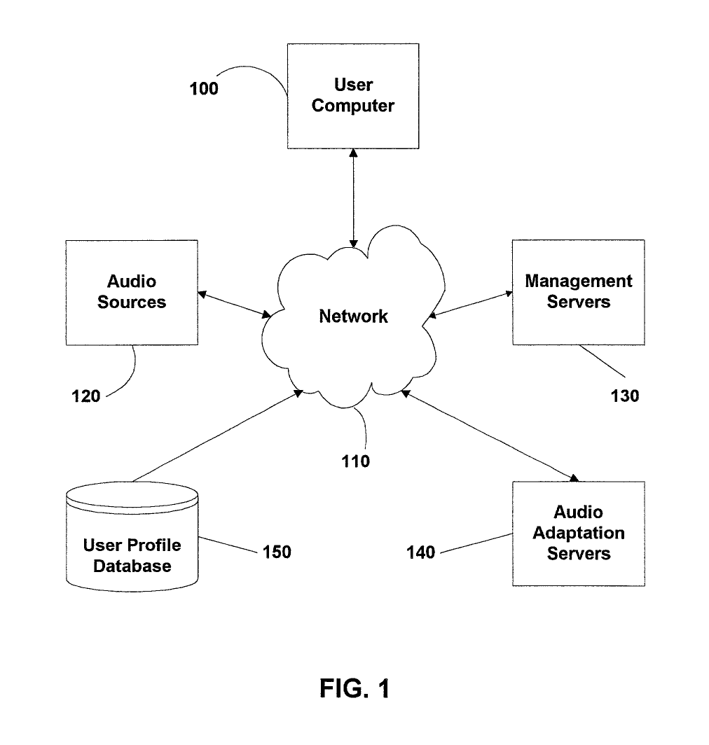

FIG. 1 depicts an exemplary operating environment of an embodiment of the invention. This includes a user's computer 100 connected to a network 110. The computer 100 preferably includes an audio output capability and the network 110 can be a local network, wide area network such as the Internet, or both. Also accessible through the network are audio sources 120, system management servers 130, audio adaptation servers 140, and user profile database 150. The audio sources 120 can be files with audio data or streaming data with audio components. Management servers 130 control the execution and communication between elements of the invention. Audio adaptation servers 140 perform the modification of audio data in response to hearing characteristics and preferences of the user. Information regarding these hearing characteristics and preferences are stored in the user profile database 150. In addition to user hearing characteristics, the user profile database 150 can include user account information and other data. The user computer 100, remote audio sources 120, management servers 130, and audio adaptation server 140 can communicate either through the network 110, or directly through other connections. Any of these elements may also reside on the same computing device. For example, the user computer 100 can also serve as an audio adaptation and management servers. If all components (120, 130, 150, and 140) reside on the user computer 100 the network 110 is not required. The user profile database 150 can be located on any of the above components or on an additional computing device but must be accessible to the audio adaptation server 140.

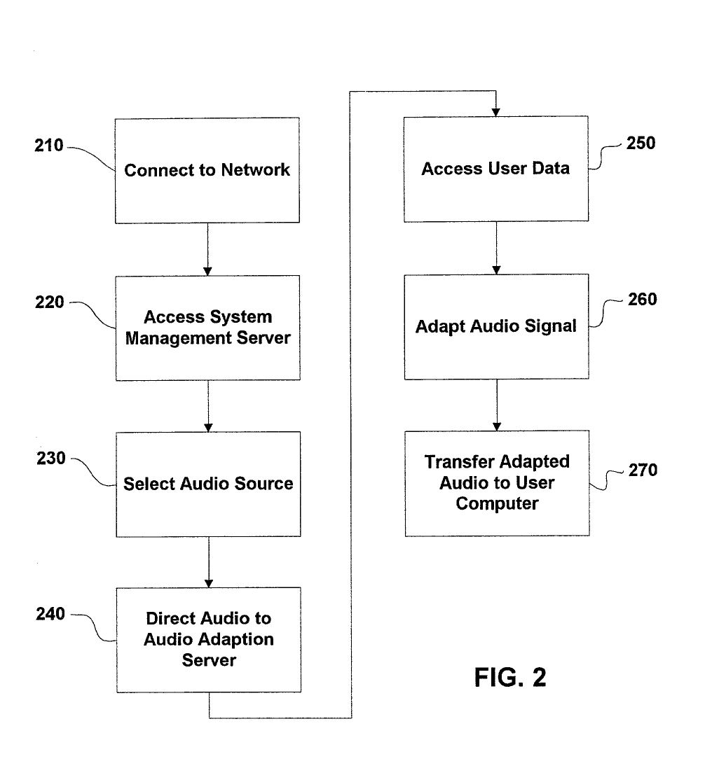

Use of the elements shown in FIG. 1 is illustrated in FIG. 2. In the first step 210 the user computer 100 connects to the network 110. If the user computer 100 is not acting as the management server 130 the next step 220 is to access a management server 130 through the network 110. This access can occur through a browser. In the third step 230 the user selects audio data at audio sources 120 and indicates their selection to the management server 130. Audio data is then directed at step 240 from the audio source 120 to an audio adaptation server 140. In the next step 250 the audio adaptation server 140 accesses the user profile database 150. This step 250 requires that the user provide identifying information and can occur prior to steps 240 or 230 if preferred. The user identification information is used to extract information specific to the user from the user profile database 150 if the database contains information related to more than one user. In step 260 the audio data is adapted based on the user's profile data. This can occur in real-time or as batch processes. In batch processes it is possible to adapt larger sections of the data and to take more time for the adaptation than in real-time. This permits adaptations of higher quality and complexity. The audio adaptation servers 140 and the management servers 130 can act as proxies for the audio sources 120. In the final step 270 the adapted audio signal is transferred to the user computer 100 (or stored on a network server). The adapted audio data can then be accessed by the user for playing using a sound system.

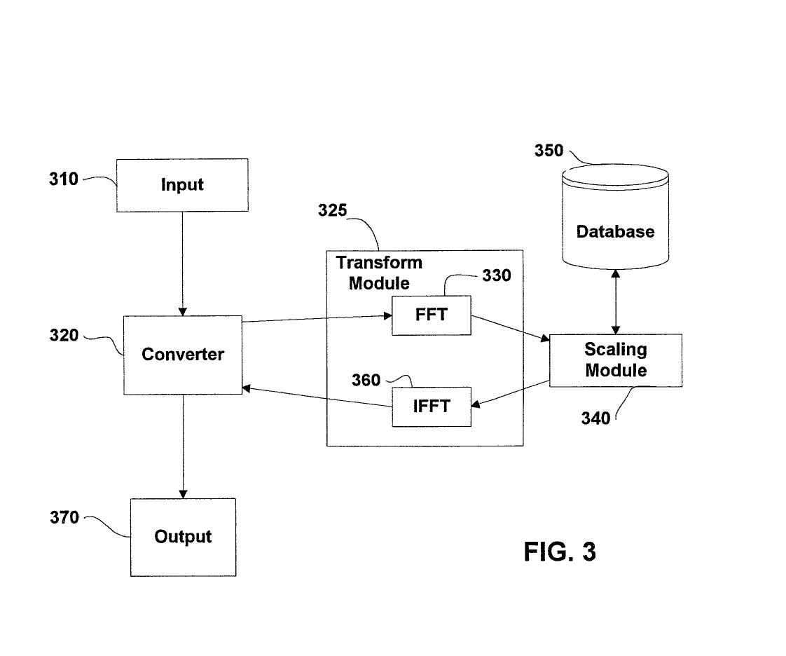

FIG. 3 depicts the components of an adaptation system, according to an embodiment of the invention. The audio data is received as input 310 to a computer program or programs. If the data is delivered in digital form, an analog to digital conversion is not required. The converter 320 then performs any necessary type (format) conversions. These can include optional conversions from any standard audio file formats such as .MP3 or .WAV. The conversion results in a digital format appropriate for input into the transform module 325 that includes procedures for executing a Fast Fourier Transform 330. The Fourier Transform procedure 330 converts the data, or a segment thereof, from the time domain to the frequency domain. In the scaling module 340 the amplitude of the signal is scaled as a function of the user's personal profile data and information relating to the user's hearing characteristics contained therein. The personal profile data is obtained from the database 350. The scaling is performed to favorably improve the user's perception of the audio signal and can include the amplification or reduction of signals at frequencies where the user has hearing impairments. After scaling the data is returned to the transform module 325 and an Inverse Fast Fourier Transform procedure 360 returns the data to the time domain. Details of performing audio adaptation using Fourier Transforms are disclosed in the prior art. The data can then optionally be converted by the converter 320 back into standard or other data types as preferred by the user. Finally, the data is delivered as output 370. The steps shown in FIG. 3 can optionally be distributed over a number of computing devices.

Operation of the transform module 325 and scaling module 340 are an example of adaptation based on user hearing data. Other known digital signal processing systems, operating in either the time or the frequency domains, can be used to achieve similar results. These operations can be substituted for modules 325 and 340 without exceeding the scope of the invention.

The adaptation process can modify the audio data to compensate for frequency dependent hearing thresholds and pain thresholds, perceived frequency shifts, and abnormal audio masking. To compensate for abnormal audio masking, adaptive signal processing is required. This processing can adapt to the signal being processed. For example, for a user whose hearing threshold is reduced for an extended period after a strong sound (abnormal temporal audio masking), the adaptive signal processing will detect the strong sound and, in response, increase the amplification component of the adaptation for an appropriate period. Adaptive signal processing can also be used to rapidly respond to changes in background sounds and thus increase signal to noise ratios.

Audio signals may be adapted for frequency shift impairments by first performing a Fast Fourier Transform, then shifting the data to higher or lower frequency in the frequency domain, and finally performing an Inverse Fast Fourier Transform. Methods of performing real-time Fourier Transforms are disclosed in Bennett or Terry.

Audio signals may be adapted for audio masking impairments by temporally adjusting the hearing threshold values, used for adaptation, in response to strong signals. For example, if user data indicates that the presence of a strong signal at 1,000 Hz raises the hearing threshold at 2,000 Hz by 20%, then the higher threshold value is used in dynamic threshold adaptation (adaptive signal processing) calculations if a strong signal is found near 1,000 Hz. If the audio masking impairment has temporal characteristics, higher threshold values may be employed for an appropriate period after the end of the strong signal. Adaptation for audio masking is only desirable when a user's masking is beyond normal parameters.

User personal preferences can include specific modification of the hearing profile, deletion, amplification, or attenuation of certain arbitrary frequency ranges, and frequency shifting of audio. The user may also set different preferences for different types of audio such as speech or music.

User hearing data can be provided to the user profile database 150 directly through the computer system on which the database 150 is located or it may be provided over a network. Delivery can be enabled by agents such as a browser, meta language file, computer program, hearing test equipment, and audiologist. Initial delivery of the data may include a user registration process that can be implemented over a network such as the Internet. The computer program and hearing test equipment can be provided over or have access to a network. In addition, hearing tests can be administered using the computer program.

The user can view and edit the data stored in the user profile database 150. The view can optionally be presented in a graphical format and the editing process can involve the use of a pointing device to select and drag points on the graph. A rapid method of data entry includes providing “normal” audio profiles and allowing the user to edit the curves until they are similar to a graph generated as the result of a hearing test.

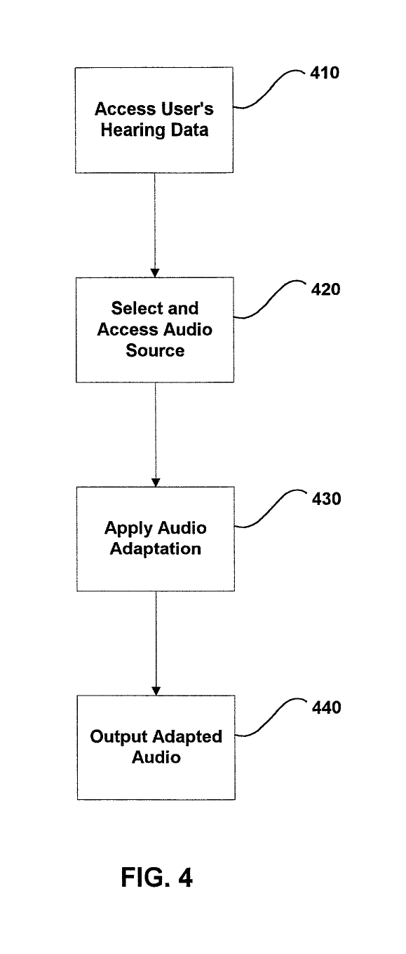

FIG. 4 further depicts steps of an embodiment of the invention. Data relating to a user's hearing ability is accessed in the first step 410. The access process can involve audio tests or the retrieval of previously stored data from the user profile database 150. In the second step 420, a source of audio data 120 is selected and data is accessed. The data may include either real-time or static (non-real-time) audio information. The order of steps 410 and 420 can be reversed. In step 430 an adaptation (FIG. 3) is applied to the audio data. The adaptation employs the data collected in step 410 to alter the audio signal for the benefit of the user. Finally, the adapted data is supplied as output in step 440. The output can be listened to immediately or stored for later use.

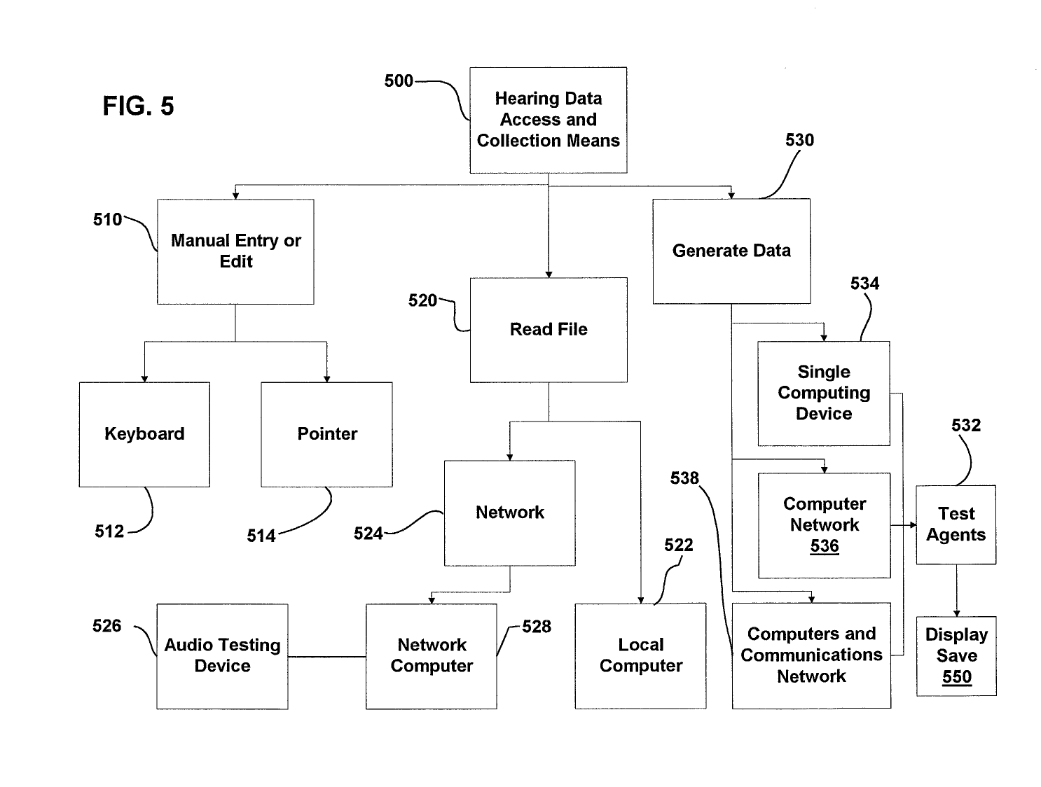

FIG. 5 illustrates several of the methods by which data can be collected and accessed in step 410 of FIG. 4. Again, the data may be related to several aspects of a user's hearing, for example, detection (hearing) thresholds as a function of frequency, pain thresholds as a function of frequency, audio masking profiles, and perceived frequency shifts. Each set of data may be collected for both the right and left ears. The elements of FIG. 5 may be used until all desired data have been collected. Various processes can also be performed in both serial and parallel manners.

Data collection means 500 includes at least three options. The first 510 is to manually enter data via a keyboard (keypad) 512 or pointing device 514, such as a computer mouse. Data can be entered in table format or a GUI can be used to manipulate graphical data displays, for example, by dragging and dropping specific points on a hearing threshold curve. Missing data can be calculated by the adaptation system using interpolation or curve fitting techniques.

The second option 520 is to retrieve data previously collected and stored in a computer file. This file can be stored on a local computer 522 or on a network computer 528 via a network 524 such as the Internet. The data can be generated either through the prior use of the elements shown in FIG. 5 or by means external to the invention such as a conventional examination by an audiologist. Delivery of data over a computer network 524 provides a number of advantages. Since a detailed audiogram can involve a large number of variables and values, these are advantages to transfering the information in digital format. This eliminates the effort and the possibilities for error associated with manual entry and/or transfer. In one embodiment, the data is transferred to a computer network from the equipment 526 used to make the hearing measurements.

The third option 530 is to generate data using computer based hearing test agents 532. These include the use of computing devices to execute computer programs that perform hearing tests. Tests can be performed by either a single computing device 534 (such as a personal computer), two or more devices connected over computer network 536 (such as the Internet), or one or more computing systems in combination with a communications network 538 such as a telephone system.

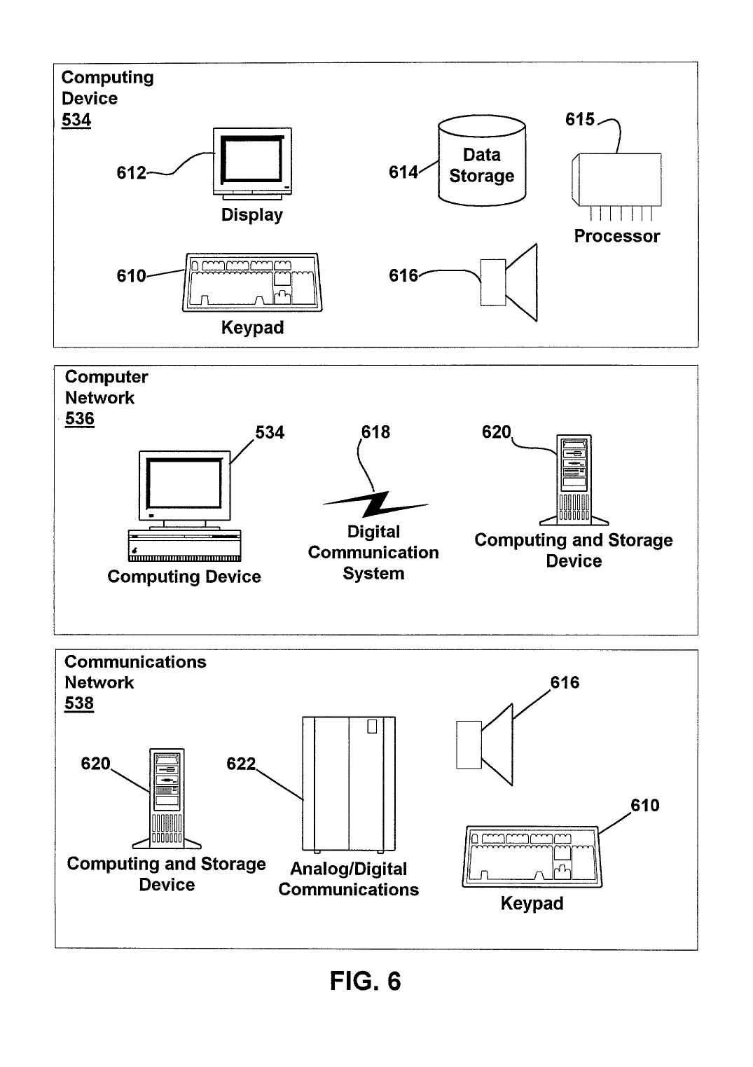

FIG. 6 shows the elements of these systems. The computing device 534 includes data entry means (keypad 610) such as keyboards, buttons, or a pointing device. It also includes display means 612, data storage means 614, digital processing means (processor 615), and audio means 616 for generating sounds. The computer network 536 includes at least one computing device 534 (in which data storage means 614 is optional), digital communications system 618, and computing and storage means (i.e. a server) 620. The communications network 538 includes at least one computing and storage means 620, a digital or analog audio communications system 622, a sound generation device 616, and data entry means (keypad 610). Sound generation device 616 and data entry means may be found in a telephone. The communications system 622 can include voice-over-Internet (IP) systems or other telephone systems.

Performing tests using specific equipment has the advantage that the audio characteristics of the equipment are included in the test. For example, testing hearing sensitivity using a telephone will generate results that take into account both a user's hearing capabilities and the frequency response of the telephone speaker. The resulting data can be ideally suited for adapting audio signals delivered to that specific telephone to a specific user. A hearing impairment is not required to attain advantage from these aspects of the invention.

The test agents 532 can include frequency hearing threshold, frequency pain threshold, audio frequency masking, audio temporal masking, and frequency shift tests. Elements of the tests can be performed in series or in parallel or in combination thereof. For example, the hearing threshold and pain threshold tests can be performed together for each specific frequency in a parallel manner or the hearing and pain tests can be serially performed separately for all frequencies. In contrast to standard hearing tests, some embodiments of the invention may not include means for detecting the absolute intensity of sound at the user's ear. However, as a feature of an embodiment of the invention, these levels can be normalized as disclosed below. All tests involve the generation of sound through a sound system. In order to develop tests for specific ears, one ear may be covered or, when possible, such as with a telephone, the sound should be applied to a specific ear. In all tests the user is asked to keep the gain on any sound system amplifiers constant.

The hearing threshold tests involve the generation of sounds of specific frequencies at progressively greater volumes. The user is asked to indicate through the input devices 512, 514, or 610 when the sound becomes audible.

The pain threshold tests involve the generation of sounds of specific frequencies at progressively greater volumes. The user is asked to indicate through the input devices 512, 514, or 610 when the sound becomes painful or when the sound becomes distorted by limitations of the sound system.

The audio frequency masking tests involve the generation of two sounds, at frequencies A and B, simultaneously. One of the sounds is gradually increased in volume and both can be temporally modulated. The user is asked to indicate, through the input devices 512, 514, or 610, when the modulated sound becomes audible. The separation between the first and second frequencies is then changed and the request is repeated. The entire process is further repeated as the first sound is varied over the audible frequency range.

The audio temporal masking tests involve the generation of two sounds within a short time period. The time period is gradually increased from an initial delay near zero seconds. The user is asked to indicate, through the input devices 512, 514, or 610, when the two distinct sounds become audible. The process is further repeated as the frequency of the sounds is varied over the audible frequency range.

During the audio masking tests it can be desirable to periodically generate only a single sound to confirm the accuracy of user input

Tests can be continued until reproducible results and sufficient data points are attained. This embodiment of the invention allows collection of a user's hearing data without a visit to an audiologist.

After the performance of test agents 532, relative results can optionally be displayed 550 to the user and changes relative to previous tests or deviations from normal results can be shown. The results are saved 550 for later use. By storing a user's hearing data on a computer network the data, and possible adaptation, is available to any device with access to the network. These devices may include telephone systems, Internet ready televisions, and computers.

In FIG. 4 step 420 an audio source is selected. In practice, any audio source may be appropriate. Audio sources can be divided into two general categories, real-time and static. Typical real-time sources include audio compact disks, streaming audio received over a network, the output of analog to digital converters, audio communication systems, and broadcasts containing an audio signal. Static sources include audio data files. These can be located on standard storage devices 614 or 620 such as hard drives, data compact disks, floppies, digital memory, or file servers and can be in any of a number of standard formats such as .WAV or .MP3. The selection of audios sources can be executed through a file manager, browser interface, or other software system.

In FIG. 4 step 430 the data collected in step 410 is used to adapt digital audio signal obtained from audio sources selected in step 420. The adaptation is intended to compensate for user hearing impairment, or deficiencies in sound sources such as 616, or both. Numerous examples of adaptation algorithms for hearing threshold and pain threshold impairments are available in the prior art. At each frequency, adaptation can be performed using an intensity curve. In Bennett this curve is defined by measured hearing threshold and pain threshold points. Terry employs the hearing threshold point and a slope.

Since the available user data can include relative intensity information, rather than absolute values as in the prior art, normalization steps may be required before adaptation algorithms are applied. To normalize hearing threshold intensity values, hearing at the frequency at which the weakest sound was detected (ƒ_{lowest}) is assumed to be normal. Threshold values at other frequencies are scaled according to the relative intensities of the measured hearing thresholds at the frequencies and at ƒ_{lowest.} Pain threshold values can be normalized in a similar manner by assuming that hearing is normal at the frequency at which the pain threshold was highest. Thus, relative values are normalized to absolute values using best-case assumptions. Using this normalized data, audio adaptation will only compensate for impairments that are frequency dependent. Users are, of course, able to adjust for non-frequency dependent impairments using standard volume control means.

Audio adaptation 430 may take place on a user's computing device or on a computer connected to a network or both. In one embodiment, adaptation takes place on a server that is part of a network such as the Internet. This server may also be the storage location for user data, or the audio source, or both. Steps in the audio adaptation process may be divided among computing devices. For example, format conversion, buffering, Fourier, or Inverse Fourier Transforms may be executed on separate systems thus reducing the computational load on any single device. Use of personal or network computers provides significantly more computing power than is available in prior art hearing aids. This allows for a substantial improvement in the quality of adaptation and allows adaptation of the entire audio frequency range. In addition, adaptation of static data files permits the use of significantly more rigorous computational techniques than is possible with the adaptation of real-time data. For example, Fourier Transforms can be calculated much more accurately and can be performed on much longer sections of the data. These factors result in an improved adaptation process.

Data relating to a user's right and left ears may be used to adapt the right and left channels of a stereo signal.

In FIG. 4 step 440 the result of the audio adaptation is supplied as output. Output may be in a digital format or, after a digital to analog conversion, be an analog signal. In a digital format, the audio information may be saved to recording media such as hard disks, compact disks, tapes, or other digital memory. Digital output may also be transmitted across computer networks, such as the Internet, or other communication systems. Analog signals may be produced in real-time or after a delay.

I claim:

1. A method of adapting audio according to a listener's auditory capability, comprising the steps of:
accessing a personal audio profile of the listener, the audio profile describing the auditory capability of the listener in relation to a plurality of audible frequencies;
accessing a digital representation of audible sound; and
creating an adapted representation of audible sound by modifying the digital representation based on the audio profile to assist the listener in perceiving the audible sound.

2. The method of claim 1, wherein the step of creating an adapted representation comprises the steps of:
converting the representation to a different data format than that in which it was accessed, creating a converted representation;
transforming the converted representation to a frequency domain vector using a Fourier transform;
scaling the frequency domain vector according to the audio profile, creating an adapted frequency domain vector;
transforming the adapted frequency domain vector to an adapted time domain sample using an inverse Fourier transform; and
converting the adapted time domain sample to a format for presentation.

3. The method of claim 2, wherein the scaling step further comprises one or more of the steps of frequency filtering, frequency shifting, frequency masking compensation, and adaptive signal processing.

4. The method of claim 1, further comprising the step of:
initiating a transmission of the adapted representation to the listener.

5. The method of claim 4 wherein the representation is accessed and the adapted representation is transmitted through a network of computers.

6. The method of claim 1 wherein the audio profile is stored in a database.

7. The method of claim 6 wherein the audio profile is provided to the database by an audio test agent through a network of computers.

8. The method of claim 1 wherein the adapted representation includes audio information representing a range of frequencies from 20 Hz to 20 kHz.

9. A system for assisting a hearing deficient user, comprising:
a database for storage of an audio profile of the user, the audio profile describing the auditory capability of the user in relation to a plurality of audible frequencies;
an adaptation engine coupled to the database for receiving an audio representation selected by the user and modifying the audio representation according to the audio profile wherein the modifying assists the user in hearing the audio representation.

10. The system of claim 9 wherein the audio representation is received over a packet-switched network of computers.

11. The system of claim 9 wherein the adaptation engine further comprises:
a converter configured to convert the audio representation from its original format into a base format, creating a converted audio representation;
a transformation module coupled to the converter configured to transform the converted audio representation into a frequency representation;
a scaling module coupled to the transformation module configured to scale the frequency representation based on the audio profile, creating a scaled representation.

12. The system of claim 11 wherein the scaled representation includes audio information representing a range of frequencies from 20 Hz to 20 kHz.

13. The system of claim 11 wherein the scaling module is further configured to scale the frequency representation by one or more of frequency filtering, frequency shifting, frequency masking compensation, and adaptive signal processing.

14. The system of claim 11 wherein the transformation module is further configured to transform the scaled representation into the base format creating a scaled converted audio representation and the converter is configured to convert the scaled converted audio representation into a presentation format creating a scaled audio representation for transmission to the user.

15. The system of claim 14 wherein the scaled audio representation is transmitted over a packet-switched network of computers.

16. The system of claim 14 wherein the scaled audio representation can be presented by a computer for listening by the user.

17. The system of claim 9 wherein the adaptation engine is located on a user computer.

18. The system of claim 9 wherein the adaptation engine is located on a computer coupled to a network, the computer being remote from the user.

19. The system of claim 9 wherein the audio profile is generated by and provided to the database by an audio testing agent through a computer network.

20. A network audio adaptation server comprising:
a memory configured to store a personal audio profile of a listener, the audio profile describing the auditory capability of the user in relation to a plurality of audible frequencies;
a proxy configured to access an audio representation selected by the listener, the audio representation being in a digital format;
a transformation module coupled to the memory and the proxy, configured to transform the audio representation into a frequency representation;
a scaling module coupled to the transformation module, configured to scale the frequency representation based on the audio profile creating a scaled representation, whereby the transformation module is further configured to transform the scaled representation into the digital format;
a transmitter for initiating delivery of the digital format scaled representation to a listener computing device via the network.

21. The server of claim 20, wherein the transformation module and the scaling module operate upon the representations in a batch process, whereby the scaled representation is of higher quality than is producible in a real-time process.

22. A machine-readable medium having embodied thereon a program, the program being executable by a machine to perform method steps for providing audio adapted according to a listener's auditory capability, the method steps comprising:
accessing a personal audio profile of the listener, the audio profile describing the auditory capability of the listener in relation to a plurality of audible frequencies;
accessing a digital representation of audible sound selected by the listener; and
creating an adapted representation of audible sound by modifying the digital representation based on the audio profile to assist the listener in perceiving the audible sound.
